(19) United States
(12) Patent Application Publication
(10) Pub. No.: US 2013/0129840 A1
(43) Pub. Date: May 23, 2013

(54) COMBINATION THERAPY USING A RUTHENIUM COMPLEX

(71) Applicant: Niiki Pharma Inc., Hoboken, NJ (US)

(72) Inventors: Hooshmand SHESHBARADARAN, Hoboken, NJ (US); Walter BERGER, Vienna (AT); Petra HEFFETER, Vienna (AT)

(73) Assignee: Niiki Pharma Inc., Hoboken, NJ (US)

(21) Appl. No.: 13/744,423

(22) Filed: Jan. 18, 2013

Related U.S. Application Data
(63) Continuation of application No. PCT/US2011/044302, filed on Jul. 18, 2011.
(60) Provisional application No. 61/365,329, filed on Jul. 18, 2010.

(51) Int. Cl. A61K 45/06; A61K 31/337; A61K 31/517; A61K 31/7068; A61K 31/44; A61K 31/17; A61K 31/513; A61K 31/704; A61K 31/436; A61K 31/404; A61K 31/4188; A61K 31/555; A61K 33/24

(52) U.S. Cl. CPC A61K 45/06; A61K 31/555; A61K 31/337; A61K 31/517; A61K 31/7068; A61K 31/513; A61K 31/704; A61K 31/436; A61K 31/404; A61K 31/4188; A61K 31/17
USPC 424/649; 514/186; 514/49; 514/34

(57) ABSTRACT

A combination therapy is disclosed for treating cancer. The method comprises administering to a cancer patient a therapeutically effective amount of trans-[tetrachlorobis(1H-indazole)ruthenate(III)] or a pharmaceutically acceptable salt thereof, and administering to the patient a therapeutically effective amount of one or more other anti-cancer agents as disclosed herein.

### RELATED APPLICATIONS

This is a continuation of PCT/US11/44302 filed on Jul. 18, 2011, which claims the priority of U.S. Provisional Application No. 61/365,329 filed on Jul. 18, 2010, the entire content of both of which being incorporated herein by reference.

### FIELD OF THE INVENTION

The present invention generally relates to method for treating cancer, and particularly to a method of treating cancer using trans-[tetrachlorobis(1H-indazole)ruthenate(III)] or a pharmaceutically acceptable salt thereof.

### BACKGROUND OF THE INVENTION

A number of ruthenium complex compounds are known in the art to be useful as anti-tumor compounds. See e.g., U.S. Pat. No. 4,843,069, PCT Publication No. WO 9736595, and US Application Publication No. 2005032801. In particular, the ruthenium complex salts indazolium trans-[tetrachlorobis(1H-indazole)ruthenate (III)] (KP1099) and sodium trans-[tetrachlorobis(1H-indazole)ruthenate (III)] (KP1339) have been shown in preclinical studies to be effective in inducing apoptosis in colon cancer cells. See e.g., Kapitza et al., J. Cancer Res. Clin. Oncol., 131(2):101-10 (2005). In addition, the compound ruthenium complex salt indazolium trans-[tetrachlorobis(1H-indazole)ruthenate (III)] (KP1019) showed some anti-cancer activities in a phase I clinical trial.

### SUMMARY OF THE INVENTION

It has been discovered that the combined use of trans-[tetrachlorobis(1H-indazole)ruthenate(III)] or a pharmaceutically acceptable salt thereof, and a number of other anti-cancer compounds creates significant synergies in the treatment of cancers. Accordingly, in a first aspect, the present invention provides a method of treating cancer in a patient in need of such treatment comprising administering to the patient, simultaneously or sequentially, a therapeutically effective amount of trans-[tetrachlorobis(1H-indazole)ruthenate(III)] or a pharmaceutically acceptable salt thereof and one or more drugs chosen from the group consisting of platinum agents (e.g., cisplatin, carboplatin, oxaliplatin, and picoplatin), taxane (e.g., docetaxel, paclitaxel), anthracyclines (e.g., doxorubicin, daunorubicin, epirubicin, idarubicin), 5-FU and prodrugs thereof (e.g., capecitabine, tegafur and S1), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab), mTOR inhibitors (e.g., everolimus, temsirolimus, ridaforolimus, sirolimus, etc.), sorafenib, regorafenib, and sunitinib.

The present invention further provides use of trans-[tetrachlorobis(1H-indazole)ruthenate(III)] or a pharmaceutically acceptable salt thereof for the manufacture of a medicament for use in combination with one or more drugs chosen from the group consisting of platinum agents (e.g., cisplatin, carboplatin, oxaliplatin, and picoplatin), taxane (e.g., docetaxel), anthracyclines (e.g., doxorubicin, daunorubicin, epirubicin, idarubicin), 5-FU and prodrugs thereof (e.g., capecitabine, tegafur and S1), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab), mTOR inhibitors (e.g., everolimus, temsirolimus, ridaforolimus, sirolimus, etc.), sorafenib, regorafenib, and sunitinib, for treating, preventing or delaying the onset of cancer.

To put it differently, the present invention provides use of one or more drugs chosen from the group consisting of platinum agents (e.g., cisplatin, carboplatin, oxaliplatin, and picoplatin), taxane (e.g., docetaxel), anthracyclines (e.g., doxorubicin, daunorubicin, epirubicin, idarubicin), 5-FU and prodrugs thereof (e.g., capecitabine, tegafur and S1), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab), mTOR inhibitors (e.g., everolimus, temsirolimus, ridaforolimus, sirolimus, etc.), sorafenib, regorafenib, and sunitinib, for the manufacture of a medicament for use in combination with trans-[tetrachlorobis(1H-indazole)ruthenate(III)] or a pharmaceutically acceptable salt thereof in treating, preventing or delaying the onset of cancer.

In yet another aspect, a kit is provided comprising in a compartmentalized container a first unit dosage form having trans-[tetrachlorobis(1H-indazole)ruthenate(III)] or a pharmaceutically acceptable salt thereof, and a second unit dosage form of one or more drugs chosen from the group consisting of platinum agents (e.g., cisplatin, carboplatin, oxaliplatin, and picoplatin), taxane (e.g., docetaxel, paclitaxel), anthracyclines (e.g., doxorubicin, daunorubicin, epirubicin, idarubicin), 5-FU and prodrugs thereof (e.g., capecitabine, tegafur and S1), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab etc.), mTOR inhibitors (e.g., everolimus, temsirolimus,ridaforolimus, sirolimus etc.), sorafenib, regorafenib, and sunitinib. Optionally, instructions on how to use the kit are included in the kit.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 shows a combination index plot illustrating the synergism between sodium trans-[tetrachlorobis(1H-indazole)ruthenate(III)] and cisplatin in the lung carcinoma cell line A549;

FIG. 2 is a combination index plot illustrating the synergism between sodium trans-[tetrachlorobis(1H-indazole)ruthenate(III)] and cisplatin in the colorectal carcinoma cell line HCT-116;

FIG. 3 is a combination index plot illustrating the synergism between sodium trans-[tetrachlorobis(1H-indazole)ruthenate(III)] and cisplatin in the gastric carcinoma cell line N87;

FIG. 4 is a combination index plot illustrating the synergism between sodium trans-[tetrachlorobis(1H-indazole)ruthenate(III)] and oxaliplatin in the colorectal adenocarcinoma cell line LoVo;

FIG. 5 is a combination index plot illustrating the synergism between sodium trans-[tetrachlorobis(1H-indazole)ruthenate(III)] and docetaxel in the prostate carcinoma cell line LNCap-1;

FIG. 6 shows an isobologram illustrating the synergism between sodium trans-[tetrachlorobis(1H-indazole)ruthenate(III)] and docetaxelin the gastric carcinoma cell line N87.Y axis is “Dose A” and X axis is “Dose B”;

FIG. 7 is a combination index plot illustrating the synergism between sodium trans-[tetrachlorobis(1H-indazole)ruthenate(III)] and 5-FU in the colorectal carcinoma cell line HCT-116;

FIG. 8 is a combination index plot illustrating the synergism between sodium trans-[tetrachlorobis(1H-indazole)ruthenate(III)] and 5-FU in the colorectal adenocarcinoma cell line LoVo;

FIG. 9 is a combination index plot illustrating the additive to synergistic activity between sodium trans-[tetrachlorobis(1H-indazole)ruthenate(III)] and 5-FU in the breast carcinoma cell line ZR-75-1;

FIG. 10 is a combination index plot illustrating the synergism between sodium trans-[tetrachlorobis(1H-indazole)ruthenate(III)] and gemcitabine in the lung carcinoma cell line A549;

FIG. 11 is a combination index plot illustrating the synergism between sodium trans-[tetrachlorobis(1H-indazole)ruthenate(III)] and gemcitabine in the pancreatic carcinoma cell line PANC-1;

FIG. 12 is a combination index plot illustrating the synergism between sodium trans-[tetrachlorobis(1H-indazole)ruthenate(III)] and sorafenib in liver carcinoma cell line Hep3B2.1-7;

FIG. 13 is a combination index plot illustrating the synergism between sodium trans-[tetrachlorobis(1H-indazole)ruthenate(III)] and sorafenib in the lung carcinoma cell line A549;

FIG. 14 is a combination index plot illustrating the synergism between sodium trans-[tetrachlorobis(1H-indazole)ruthenate(III)] and doxorubicinin the liver carcinoma cell line Hep 3B 2.1-7;

FIG. 15 is a combination index plot illustrating the synergism between sodium trans-[tetrachlorobis(1H-indazole)ruthenate(III)] and erlotinib in the lung carcinoma cell line A549;

FIG. 16 is a graph showing the synergism between sodium trans-[tetrachlorobis(1H-indazole)ruthenate(III)] and erlotinib in the cervix carcinoma cell line KB-3-1 (X axis: sodium trans-[tetrachlorobis(1H-indazole)ruthenate(III)] concentration (μM), Y axis: O.D. (optical density), E: erlotinib);

FIG. 17 is a graph showing the synergism between sodium trans-[tetrachlorobis(1H-indazole)ruthenate(III)] and erlotinib in the liver carcinoma cell line Hep3B (X axis: sodium trans-[tetrachlorobis(1H-indazole)ruthenate(III)] concentration (μM), Y axis: O.D. (optical density), E: erlotinib);

FIG. 18 is a graph showing the synergism between sodium trans-[tetrachlorobis(1H-indazole)ruthenate(III)] and BCNU in the liver carcinoma cell line Hep3B (X axis: sodium trans-[tetrachlorobis(1H-indazole)ruthenate(III)] concentration (μM), Y axis: O.D. (optical density));

FIG. 19 is a graph showing the synergism between sodium trans-[tetrachlorobis(1H-indazole)ruthenate(III)] and BCNU in the cervix carcinoma cell line KB-3-1 (X axis: sodium trans-[tetrachlorobis(1H-indazole)ruthenate(III)] concentration (μM), Y axis: O.D. (optical density));

FIG. 20 is a graph showing the synergism between sodium trans-[tetrachlorobis(1H-indazole)ruthenate(III)] and sunitinib in the liver carcinoma cell line Hep3B (X axis: sodium trans-[tetrachlorobis(1H-indazole)ruthenate(III)] concentration (μM), Y axis: O.D. (optical density));

FIG. 21 is a graph showing the synergism between sodium trans-[tetrachlorobis(1H-indazole)ruthenate(III)] and temozolomide in the liver carcinoma cell line Hep3B (X axis: sodium trans-[tetrachlorobis(1H-indazole)ruthenate(III)] concentration (μM), Y axis: O.D. (optical density));

FIG. 22 is a graph showing the synergism between sodium trans-[tetrachlorobis(1H-indazole)ruthenate(III)] and temozolomide in the cervix carcinoma cell line KB-3-1 (X axis: sodium trans-[tetrachlorobis(1H-indazole)ruthenate(III)] concentration (μM), Y axis: O.D. (optical density));

FIG. 23 is a graph showing the synergism between sodium trans-[tetrachlorobis(1H-indazole)ruthenate(III)] and sorafenib in the hepatoma cell line Hep3B (X axis: sodium trans-[tetrachlorobis(1H-indazole)ruthenate(III)] concentration (μM), Y axis: O.D. (optical density));

FIG. 24 is a graph showing the synergism between sodium trans-[tetrachlorobis(1H-indazole)ruthenate(III)] and sorafenib in the hepatoma cell line HepG2 (X axis: sodium trans-[tetrachlorobis(1H-indazole)ruthenate(III)] concentration (μM), Y axis: O.D. (optical density));

FIG. 25 is a graph showing the synergism between sodium trans-[tetrachlorobis(1H-indazole)ruthenate(III)] and sorafenib in the lung carcinoma cell line VL-8 (X axis: sodium trans-[tetrachlorobis(1H-indazole)ruthenate(III)] concentration (μM), Y axis: O.D. (optical density));

FIG. 26 is a graph showing the synergism between sodium trans-[tetrachlorobis(1H-indazole)ruthenate(III)] and sorafenib in the lung carcinoma cell line A549 (X axis: sodium trans-[tetrachlorobis(1H-indazole)ruthenate(III)] concentration (μM), Y axis: O.D. (optical density));

FIG. 27 is a graph showing the synergism between sodium trans-[tetrachlorobis(1H-indazole)ruthenate(III)] and sorafenib in the mesothelioma cell line P31 (X axis: sodium trans-[tetrachlorobis(1H-indazole)ruthenate(III)] concentration (μM), Y axis: O.D. (optical density));

FIG. 28 is a graph showing the synergism between sodium trans-[tetrachlorobis(1H-indazole)ruthenate(III)] and sorafenib in the melanoma cell line VM-1 (X axis: sodium trans-[tetrachlorobis(1H-indazole)ruthenate(III)] concentration (μM), Y axis: O.D. (optical density), S: sorafenibs);

FIG. 29 is a graph showing the synergism between sodium trans-[tetrachlorobis(1H-indazole)ruthenate(III)] and sorafenib in the colon cancer cell line SW480 (X axis: sodium trans-[tetrachlorobis(1H-indazole)ruthenate(III)] concentration (μM), Y axis: O.D. (optical density), S: sorafenib);

FIG. 30 is a graph illustrating that the combination between sodium trans-[tetrachlorobis(1H-indazole)ruthenate(III)] and sorafenib in the Hep3B liver carcinoma xenograft model yields long term survival (Y-axis: % survival; X-axis: days);

FIG. 31 is a combination index plot illustrating the synergism between sodium trans-[tetrachlorobis(1H-indazole)ruthenate(III)] and everolimus in the neuroendocrine tumor cell line MKL-1.

### DETAILED DESCRIPTION OF THE INVENTION

The present invention provides a method of treating cancer by a combination therapy. The method comprises treating a cancer patient in need of treatment with a therapeutically effective amount of trans-[tetrachlorobis(1H-indazole)ruthenate(III)] or a pharmaceutically acceptable salt thereof, as well as one or more drugs chosen from the group consisting of platinum agents (e.g., cisplatin, carboplatin, oxaliplatin, and picoplatin), taxane (e.g., docetaxel and paclitaxel), anthracyclines (e.g., doxorubicin, daunorubicin, epirubicin, idarubicin), 5-FU and prodrugs thereof (e.g., capecitabine, tegafur and S1), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab), mTOR inhibitors (e.g., everolimus, temsirolimus, ridaforolimus, sirolimus, etc.), sorafenib, regorafenib, and sunitinib. As used herein, the term “pharmaceutically acceptable salts” refers to the relatively non-toxic, organic or inorganic salts of the active compounds, including inorganic or organic salts of the compound. Exemplary salts of trans-[tetrachlorobis(1H-indazole)ruthenate(III)] include indazolium salt (e.g.,indazolium trans-[tetrachlorobis(1H-indazole)ruthenate(III)]), and alkali metal salts (e.g., sodium trans-[tetrachlorobis(1H-indazole)ruthenate(III)]), etc. As used herein, the phrase “treating . . . with . . . ” means either administering a compound to the patient or causing the formation of a compound inside the patient.

In some embodiments, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) one or more anti-cancer agents chosen from the group consisting of platinum agents (e.g., cisplatin, carboplatin, oxaliplatin, and picoplatin), taxane (e.g., docetaxel and paclitaxel),anthracyclines (e.g., doxorubicin, daunorubicin, epirubicin, idarubicin), 5-FU and prodrugs thereof (e.g., capecitabine, tegafur and S1), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab), mTOR inhibitors (e.g., everolimus, temsirolimus, ridaforolimus, sirolimus, etc.), sorafenib, regorafenib, and sunitinib. To put it differently, in accordance with this embodiment, the method comprises administering a therapeutically effective amount of a pharmaceutically acceptable salt of trans-[tetrachlorobis(1H-indazole)ruthenate(III)] (e.g., an alkali metal salt such as sodium salt) to a cancer patient who is under treatment of the one or more other anti-cancer agents provided above, or administering a therapeutically effective amount of such one or more other anti-cancer agents provided above to a cancer patient who is under treatment of a pharmaceutically acceptable salt of trans-[tetrachlorobis(1H-indazole)ruthenate(III)].

A variety of cancers can be treated with the method of the present invention, including, but not limited to, brain cancer (e.g., astrocytoma such as glioblastoma), breast cancer, ovarian cancer, cervical cancer, gastric cancer, esophageal cancer, lung cancer (NSCLC and small cell lung cancer), colorectal cancer, liver cancer (e.g., hepatocellular carcinoma), melanoma, pancreatic cancer, neuroendocrine tumors, prostate cancer, renal cancer, endometrial cancer, and sarcoma.

In one embodiment, colorectal cancers such as colon carcinoma are treated with the combination method of the present invention. In another embodiment, liver cancers such as hepatocellular carcinoma are treated with the combination method of the present invention. In another embodiment, the combination therapy method of the present invention is used to treat melanoma. In another embodiment, lung cancer (e.g. NSCLC and SCLC) is treated with the combination therapy method. In yet another embodiment, gastroesophageal cancer (e.g., gastric cancer, esophageal cancer) is treated with the combination therapy. In another embodiment, breast or ovarian cancer is treated with the combination therapy. In yet another embodiment, prostate cancer is treated with the combination therapy. In yet another embodiment, the combination therapy is applied to cervical or endometrial cancer. In yet another embodiment, kidney cancer is treated using the combination therapy method of the present invention. In yet another embodiment, the combination therapy is applied to pancreatic cancer. In another embodiment, the combination therapy is applied to neuroendocrine tumors.

Thus, in these various embodiments, in accordance with the present invention, a patient having cancer is identified or diagnosed, and the patient is treated with a therapeutically effective amount of trans-[tetrachlorobis(1H-indazole)ruthenate(III)] or a pharmaceutically acceptable salt thereof, and a therapeutically effective amount of the one or more anti-cancer agents provided above.

In one embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) a platinum agent such as cisplatin, carboplatin and oxaliplatin. In specific embodiments, the method is used for the treatment of colorectal cancer, lung cancer, or gastroesophageal cancer (e.g., gastric cancer or esophageal cancer). In other specific embodiments, the method is used for treating ovarian cancer, small cell lung cancer, testicular cancer, bladder carcinoma. In other specific embodiments, the method is used for treating head and neck cancer, and brain tumors. In one specific embodiment, the combination of (1) a pharmaceutically acceptable salt of trans-[tetrachlorobis(1H-indazole)ruthenate(III)] (e.g., an alkali metal salt such as sodium salt) and (2) oxaliplatin is used for the treatment of colorectal cancer. In one specific embodiment, the combination of (1) a pharmaceutically acceptable salt of trans-[tetrachlorobis(1H-indazole)ruthenate(III)] (e.g., an alkali metal salt such as sodium salt) and (2) a platinum agent (e.g., cisplatin, carboplatin and oxaliplatin) is used for the treatment of lung cancer. In another specific embodiment, the combination of (1) a pharmaceutically acceptable salt of trans-[tetrachlorobis(1H-indazole)ruthenate(III)] (e.g., an alkali metal salt such as sodium salt) and (2) a platinum agent (e.g., cisplatin, carboplatin and oxaliplatin) is used for the treatment of gastroesophageal cancer.

In another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) a taxane (e.g., docetaxel, paclitaxel). In some specific embodiments, the method is used for the treatment of prostate cancer, gastroesophageal cancer (e.g., gastric cancer) and lung cancer (e.g., non-small cell lung cancer). In some specific embodiments, the method comprises administering,simultaneously or sequentially, to a patient (1) a pharmaceutically acceptable salt of trans-[tetrachlorobis(1H-indazole)ruthenate(III)] and (2) docetaxel for the treatment of breast cancer, lung cancer, prostate cancer, gastroesophageal cancer, or head and neck cancer. In some other specific embodiments, the method comprises administering to a patient (1) a pharmaceutically acceptable salt of trans-[tetrachlorobis(1H-indazole)ruthenate(III)] and (2) paclitaxel for the treatment of breast cancer, ovarian cancer, lung cancer, head and neck cancer, gastric cancer, esophagus cancer, bladder cancer, endometrial cancer, or cervical cancer.

In another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) an anthracycline (e.g., doxorubicin, daunorubicin, epirubicin, idarubicin, particularly doxorubicin). In some specific embodiments, the combination comprising (1) a pharmaceutically acceptable salt of trans-[tetrachlorobis(1H-indazole)ruthenate(III)] (e.g., an alkali metal salt such as sodium salt), and (2) doxorubicin is applied to treat liver cancer (e.g., hepatocellular carcinoma).

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) 5-fluorouracil or a prodrug thereof (e.g., capecitabine, tegafur and S1). In specific embodiments, the method is used for the treatment of colorectal cancer or breast cancer or pancreatic cancer.

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) gemcitabine. In some specific embodiments, the cancer treated is pancreatic cancer, lung cancer, bladder cancer or breast cancer.

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) an EGFR inhibitor. In some specific embodiments, the method is applied to the treatment of lung cancer (e.g., NSCLC), pancreatic cancer, cervical cancer colorectal cancer, or liver cancer (particular hepatocellular carcinoma). EGFR inhibitors are well known in the art, including, but not limited to, small molecule EGFR inhibitors (e.g., erlotinib, gefitinib, afatinib), and EGFR antibodies (cetuximab, panitumumab, nimotuzumab, necitumumab, etc.).

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) sorafenib or regorafenib. In specific embodiments, the cancer treated is liver cancer (e.g., hepatocellular carcinoma), lung cancer (e.g., NSCLC), colorectal cancer or melanoma.

In another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) sunitinib. In specific embodiments, the combination is used to treat liver cancer (e.g., hepatocellular carcinoma). In other specific embodiments, the combination is used to treat renal cell carcinoma, gastrointestinal stromal tumor, and neuroendocrine tumors.

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) temozolomide. In specific embodiments, the combination is used for the treatment of liver cancer, brain cancer (e.g., glioblastoma) or melanoma.

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) BCNU. In some specific embodiments, the combination therapy is used for the treatment of liver cancer or cervical cancer or brain cancer.

In yet another embodiment, the method of treating cancer comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) one or more mTOR inhibitors. Examples of mTOR inhibitors include, but not limited to, e.g., everolimus, temsirolimus, ridaforolimus, sirolimus etc. In some specific embodiments, the combination therapy is used for the treatment of neuroendocrine tumors (NET), kidney cancer, astrocytoma, breast cancer, gastric cancer, or hepatocellular carcinoma. In some specific embodiments, the combination therapy comprises administering to a cancer patient in need of treatment, simultaneously or sequentially, a therapeutically effective amount of (1) a pharmaceutically acceptable salt of trans-[tetrachlorobis(1H-indazole)ruthenate(III)] (e.g., an alkali metal salt such as sodium salt), and (2) everolimus for treating neuroendocrine tumors (NET).

Alkali metal salts of trans-[tetrachlorobis(1H-indazole)ruthenate(III)] are known in the art and disclosed in e.g., European Patent No. EP 0835112 B1, and can be made in any methods known in the art. For example, PCT Publication No. WO/2008/154553 discloses an efficient method of making sodium trans-[tetrachlorobis(1H-indazole)ruthenate(III)]. Indazolium salt of trans-[tetrachlorobis(1H-indazole)ruthenate(III)] is disclosed in U.S. Pat. No. 7,338,946.

The trans-[tetrachlorobis(1H-indazole)ruthenate(III)] or a pharmaceutically acceptable salt thereof and the one or more other anti-cancer agents can be administered at about the same time, or separately according to their respective dosing schedules. When administered at about the same time, the trans-[tetrachlorobis(1H-indazole)ruthenate(III)] or a pharmaceutically acceptable salt thereof can be administered in the same pharmaceutical composition or in separate dosage unit forms. Trans-[tetrachlorobis(1H-indazole)ruthenate(III)] and pharmaceutically acceptable salts thereof, such as sodium trans-[tetrachlorobis(1H-indazole)ruthenate(III)] and indazolium trans-[tetrachlorobis(1H-indazole)ruthenate(III)] can be administered through intravenous injection or any other suitable means at a dosing of from 0.1 mg to 1000 mg per kg of body weight of the patient based on total body weight. The active ingredients may be administered at once, or may be divided into a number of smaller doses to be administered at predetermined intervals of time, e.g., once daily or once every two days. See e.g., Hartinger et al., J. Inorg. Biochem., 100:891-904 (2006). Injectable forms are generally known in the art, e.g., in buffered solution or suspension.

The other anti-cancer agents used in combination with a salt of trans-[tetrachlorobis(1H-indazole)ruthenate(III)] can be administered through a route and at an amount generally recommended by their manufacturers or known in the art, e.g., as provided in the prescribing information sheet or product package insert as approved by relevant regulatory authorities, or varied therefrom, e.g., by one order of magnitude as clinicians see fit to accommodate specific patient situations.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with another aspect of the present invention, a pharmaceutical kit is provided comprising in a compartmentalized container (1) a unit dosage form of a pharmaceutically acceptable salt of trans-[tetrachlorobis(1H-indazole)ruthenate (III)], such as sodium trans-[tetrachlorobis(1H-indazole)ruthenate(III)] and indazolium trans-[tetrachlorobis(1H-indazole)ruthenate(III)]; and (2) a unit dosage form of at least one (one, two, or more) anti-cancer agent chosen from the group consisting of platinum agents (e.g., cisplatin, carboplatin, oxaliplatin, and picoplatin), taxane (e.g., docetaxel, paclitaxel), anthracyclines (e.g., doxorubicin, daunorubicin, epirubicin, idarubicin), 5-FU and prodrugs thereof (e.g., capecitabine, tegafur and Si), nitrosourea compounds (e.g., carmustine (BCNU), lomustine (CCNU), semustine, ethylnitrosourea (ENU) and streptozotocin), gemcitabine, temozolomide, EGFR inhibitors (e.g., erlotinib, gefitinib, cetuximab, panumutimab), mTOR inhibitors (e.g., everolimus, temsirolimus, ridaforolimus, sirolimus etc.), sorafenib, regorafenib, and sunitinib. As will be apparent to a skilled artisan, the amount of a therapeutic compound in the unit dosage form is determined by the dosage to be used on a patient in the method of the present invention. In the kit, a pharmaceutically acceptable salt trans-[tetrachlorobis(1H-indazole)ruthenate(III)] can be in lyophilized form in an amount of, e.g., 25 mg, in an ampoule. The other anti-cancer agents to be used in the combination therapy and included in the kit can be in any dosage form generally known or used in the art, e.g., tablet, capsule, a lyophilized form for reconstitution of an injectable form, etc. Optionally, the kit further comprises instructions for using the kit in the combination therapy method in accordance with the present invention.

### EXAMPLE 1

Cell Culture: Human tumor cell lines including A549, HCT-116, Hep 3B2.1-7, LNCap clone FGC, LoVo, N87, PANC-1 and ZR-75-1 were obtained from the American Type Culture Collection (ATCC) or the UNC Lineberger Comprehensive Cancer Center. The MKL-1 human neuroendocrine skin carcinoma cell line was obtained from the ECACC (European Collection of Cell Cultures). Cell cultures were established using standard in vitro culture methods and supplier recommended media and supplements in 175 cm² Greiner® or Corning® tissue culture-treated flasks. All cell cultures were incubated in a humidified 37° C., 5% CO₂, 95% air environment. The cells were sub-cultured regularly to maintain log phase growth.

On the day of EC₅₀ plate seeding, the cells for each line were processed and seeded into 96-well cell culture-treated plates one cell line at a time. The cells were removed from their culture flasks using trypsin solution pooled in a sterile conical tube and centrifuged at 350×g for 5 minutes at room temperature. For MKL-1 cells in suspension, the cells did not require trypsinization.

The cell suspensions were diluted (based on live cell counts) using complete media to yield a final suspension density (cells/ml) based on previously determined seeding densities for each cell line for a 72 hour 96-well plate assay. The tissue culture treated plates for EC₅₀ testing were seeded at a density specified below in Table 1 and incubated overnight at 37° C. in a 5% CO₂, 95% air humidified atmosphere to allow the cells to attach.

TABLE 1
Seeding Density for EC₅₀ Assay

| | | Cells/well |
|---|---|---|
| Cell Line | Type | (×10³) |
| A549 | lung cancer | 2.5 |
| HCT-116 | colorectal cancer | 8.0 |
| Hep 3B2.1-7 | hepatocellular | 6.0 |
| | carcinoma | |
| LNCaP | prostate cancer | 4.0 |
| LoVo | colorectal cancer | 12.0 |
| N87 | gastric cancer | 20.0 |
| PANC-1 | pancreatic cancer | 6.0 |
| ZR-75-1 | breast cancer | 3.0 |
| MKL-1 | human neuroendocrine | 34 |
| | skin carcinoma | |

Test Agent Preparation: For each single agent or combination of test agents, the top concentration mixture (2× final treatment concentration) was made in sterile 1.5 ml microcentrifuge tubes and then directly transferred to the first well of the treatment dilution plates. A 200 mM stock solution of sodium trans-[tetrachlorobis(1H-indazole)ruthenate(III)] (“test drug”) was made using 500 μl of 100% dimethyl sulfoxide (DMSO). An aliquot of the 200 mM stock solution was used to also make a 40 mM stock solution in 100% DMSO (10 μl of 200 mM stock+40 μl DMSO for the N87 cell line).

5-Fluororuracil was manufactured by TEVA Parenteral Medicines and supplied in vials at a concentration of 50 mg/ml (384.4 mM) in aqueous solution.

Cisplatin was obtained from Sigma-Aldrich, and a 4 mM stock solution of cisplatin was made using 0.9% saline and stored at −20° C. After thawing, the 4 mM stock solution was diluted 2× using complete media to yield a 2 mM solution in the first well of a 96-well dilution plate for the positive control test plate wells. This was then serially diluted 1:4 in complete media across nine wells for a total of ten concentrations ranging from 2,000-0.008 μM. The 4 mM stock solution was also diluted for use as a single standard agent and in combination with sodium trans-[tetrachlorobis(1H-indazole)ruthenate(III)].

Docetaxel manufactured by Fluka was weighed out (1.6 mg) and a 2,000 μM solution was made by adding 0.990 ml 100% DMSO and intermittently vortexing for 1-15 seconds. This was further diluted in 100% DMSO to make a 40 μM stock solution (10 μl of 2,000 μM docetaxel+490 μl DMSO).

8.2 mg of Erlotinib (from LC Laboratories) was weighed out and a 50 mM cloudy, white suspension was made by adding 0.382 ml 100% DMSO and intermittently vortexing for 15-30 seconds.

5.8 mg of Gemcitabine (manufactured by Eli Lilly and Company) was weighed out and a 50 mM clear, colorless stock solution was made by adding 188 μl of sterile water. This was further diluted 1,000× in complete media to yield a 50 μM stock solution (10 μl of 50 mM gemcitabine+9.990 ml media).

Sorafenib was obtained from LC Laboratories and a 100 mM stock solution was made by adding 0.188 ml of 100% DMSO to 12.0 mg sorafenib.

Everolimus was obtained from LC Laboratories and a 48 mM clear, colorless stock solution was made by adding 117 μl of 100% DMSO to 5.4 mg of everolimus.

EC₅₀ Assay: The antiproliferative activity of the test agents was evaluated using the MTT Cell Proliferation Assay Kit (ATCC catalog #30-1010K). The MTT assay is based on the reduction of yellow tetrazolium MTT (3-(4,5-dimethylthiazolyl-2)-2,5-diphenyltetrazolium bromide) by metabolically active cells forming purple formazan crystals. The purple formazan is solubilized with detergent and quantified spectrophotometrically at 570 nm.

Cells in the log phase of growth were seeded at the indicated densities listed in Table 1 above into 96-well culture treated plates in 0.1 mL of complete media in all wells except for one column reserved for the media only control. The cells (except for the MKL-1 cells) were allowed to attach during an overnight incubation prior to treating with test agents. Test agents were serially diluted in complete culture media (+1% DMSO where appropriate) and added to each well in a volume of 0.1 mL for a total final volume of 0.2 mL/well (0.5% DMSO final, where used). Cells were exposed to test agents for 72 hours. Following the exposure to test agents, 0.1 mL of culture supernatant was carefully removed from all wells of each plate and 0.01 mL of MTT reagent was added to each well. The plates were returned to the incubator for four hours. Following the incubation period, kit supplied detergent reagent (0.1 mL) was added to all wells. The plates were wrapped in plastic wrap to prevent evaporation and allowed to sit at room temperature in the dark overnight. The absorbance at 570 nm was measured the following day using a SpectraMAX Plus plate reader (Molecular Devices).

Absorbance values were converted to Percent of Control and plotted against test agent concentrations for EC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration vs. Percent of Control were analyzed using the 4-parameter equation to obtain EC₅₀ values and other parameters that describe the sigmoidal dose response curve.

Combination data was analyzed using CompuSyn® software to calculate Combination Index (CI) values to assess synergy. The Fractional Affect (Fa) was calculated from the Percent of Control (from SoftMax® Pro) using the formula: 1−(Percent Control/100). The dosage, fractional affect and molar ratio of compounds tested in combination were entered into the CompuSyn® software for evaluation of the presence/absence of synergy. CompuSyn® assigns a Combination Index (CI) value which rates the level of compounds' effect on proliferation. CI values below 1 indicate the presence of synergy and CI values above 1 indicate antagonism. CI values close to 1 indicate an additive affect. See Chou, PHARMACOL. REV., 58(3):621-81(2006). Table 2 below summarizes the CI values of the synergistic combinations.

TABLE 2
Combination Index Values

| | | Combination Index |
|---|---|---|
| Combination | Cell Line | (CI) Values* |
| Test drug + cisplatin | A549 | 0.1729 |
| | HCT-116 | 0.6872 |
| | N87 | 0.7575 |
| Test drug + oxaliplatin | LoVo | 0.219 |
| Test drug + docetaxel | LNCaP | 0.5435 |
| | N87 | 0.6954 |
| Test drug + 5-fluorouracil | HCT-116 | 0.3608 |
| | LoVo | 0.5975 |
| | ZR-75-1 | 0.6516 |
| Test drug + gemcitabine | A549 | 0.6472 |
| | PANC-1 | 0.8952 |
| Test drug + sorafenib | Hep3B2.1-7 | 0.5361 |
| | A549 | 0.8469 |
| Test drug + doxorubicin | Hep3B2.1-7 | 0.252 |
| Test drug + erlotinib | A549 | 0.5093 |
| Test drug + everolimus | MKL-1 | 0.354 |

*0.1-0.90 = Synergism; 0.90-1.10 = Additive; 1.10-10 = Antagonism.

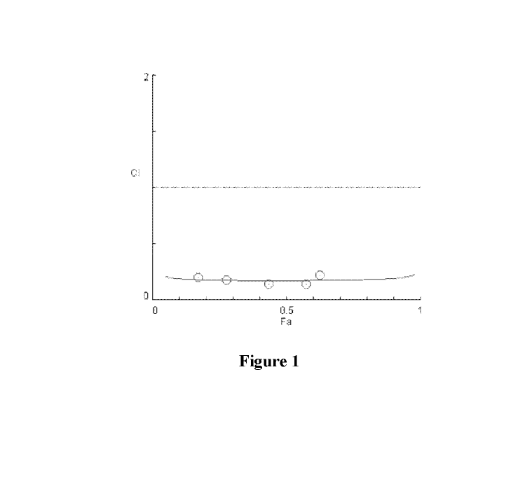

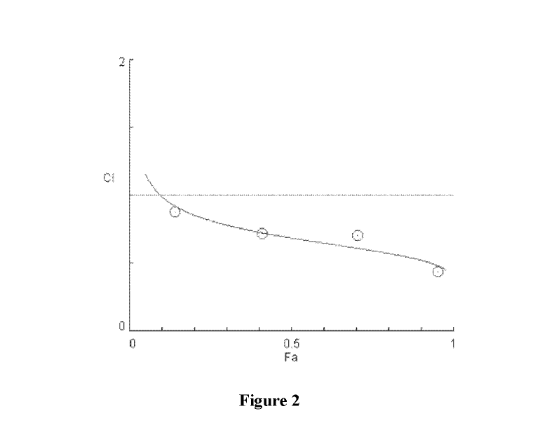

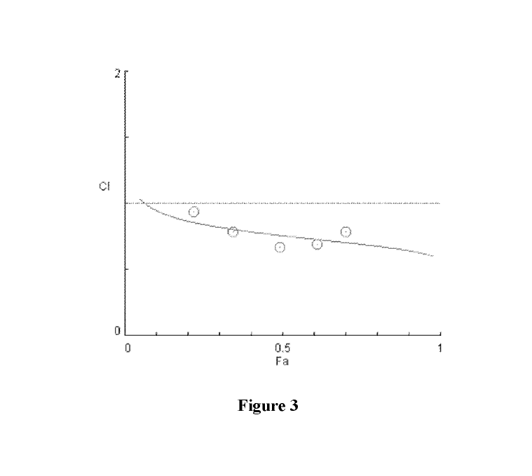

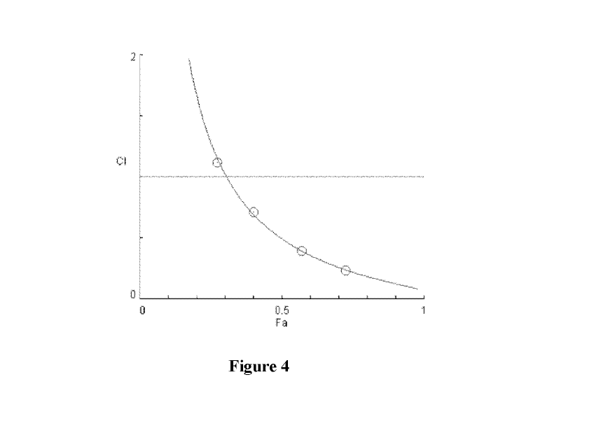

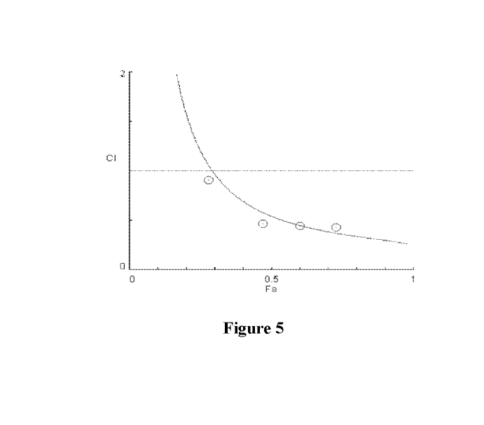

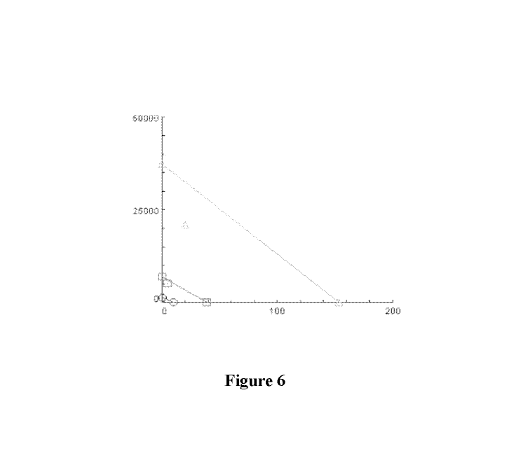

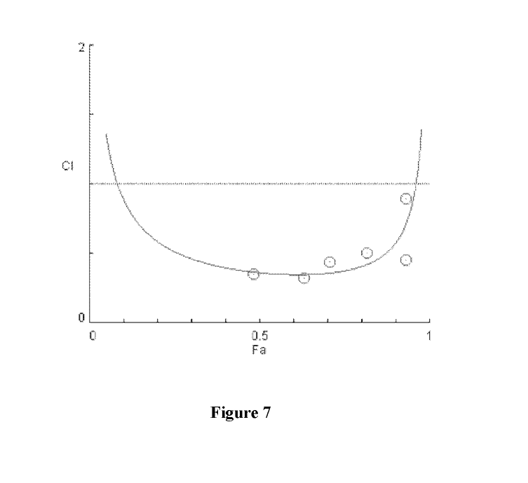

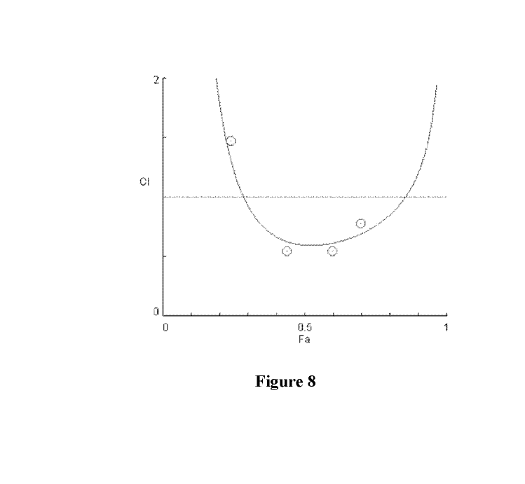

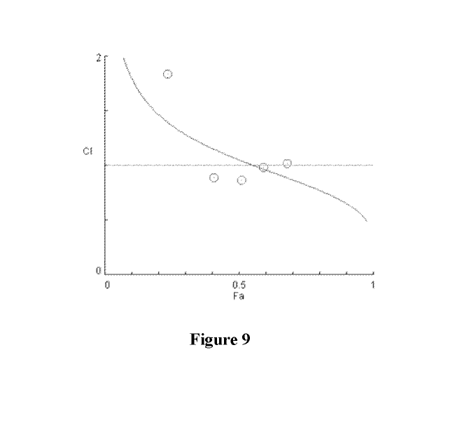

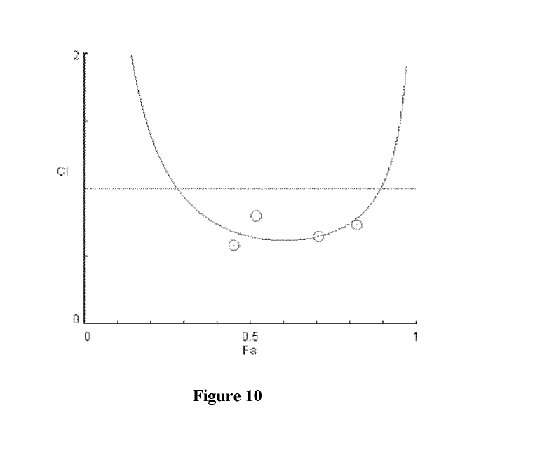

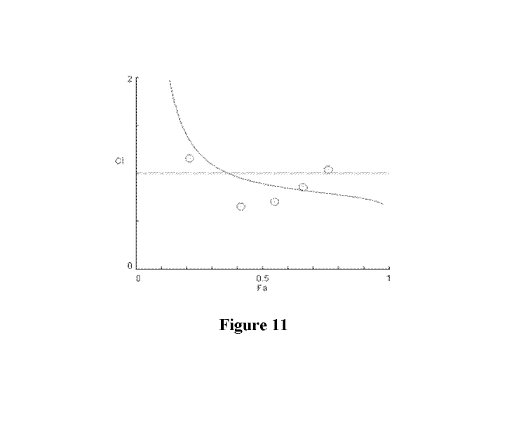

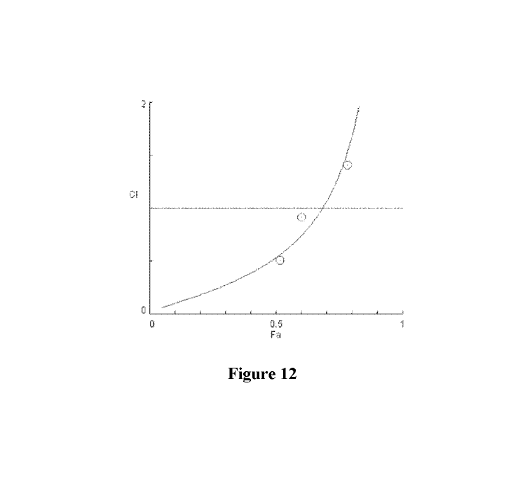

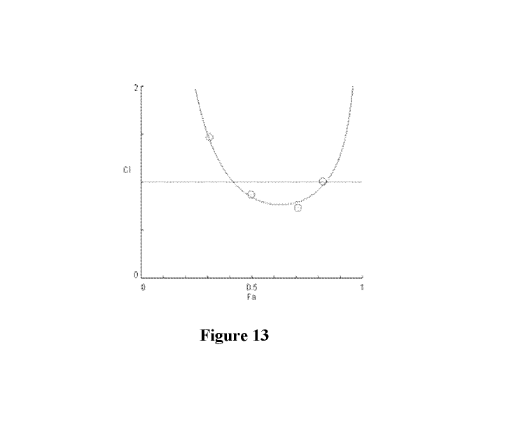

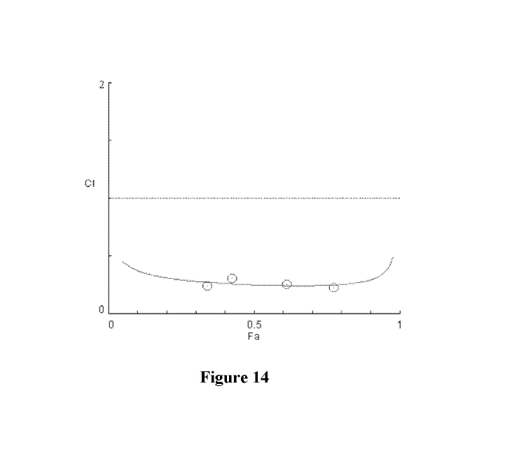

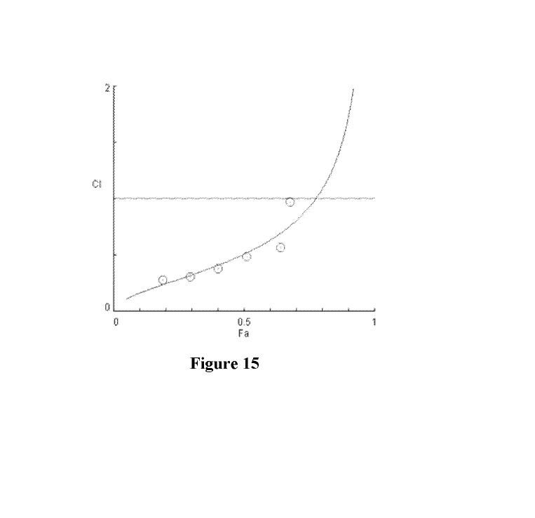

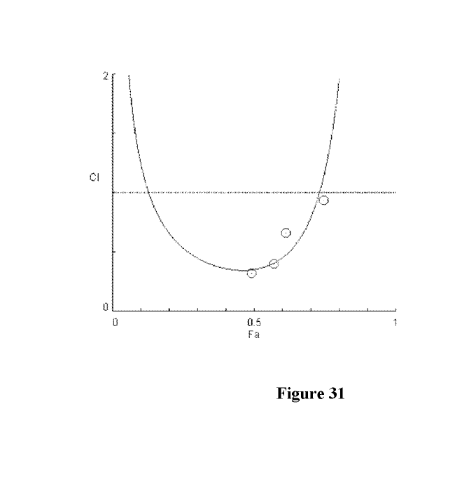

Using CompuSyn software, the combination index (CI) values at different (fa)x (fraction affected) were generated, and the entire spectrum of CIs at different fa values were simulated. The synergism is further illustrated in the isobologram combination index plots in FIGS. 1-15, 31. Note that both the Fa and the CI for the x- and y-axes are dimensionless quantities. Points under the dashed line are synergistic.

### EXAMPLE 2

Cell Culture: The hepatocellular carcinoma cell line Hep3B (from ATCC) was grown in RMPI 1640 supplemented with 10% fetal bovine serum. The epidermal carcinoma-derived cell line KB-3-1 was grown in RPMI 1640+10% FCS. See Shen et al., J. Biol. Chem., 261:7762-7770 (1986).

Cytotoxicity Assays: Cells were plated (2×10³ cells in 100 μl/well) in 96-well plates and allowed to recover for 24 hours. Drugs were added in another 100 μl growth medium and cells exposed for 72 hours. The proportion of viable cells was determined by MTT assay following the manufacturer's recommendations (EZ4U, Biomedica, Vienna, Austria).

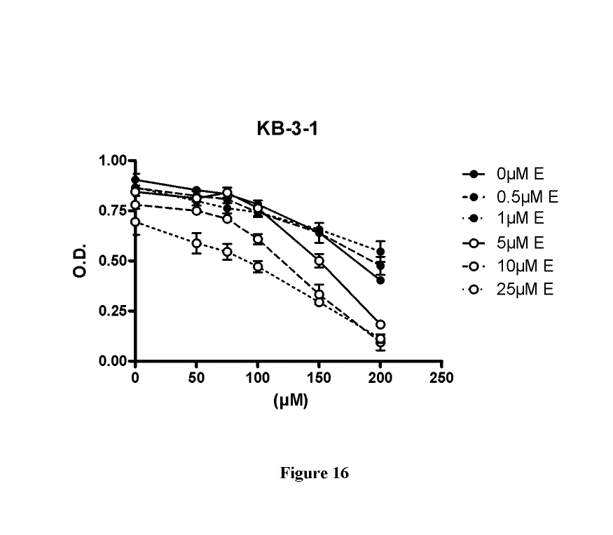

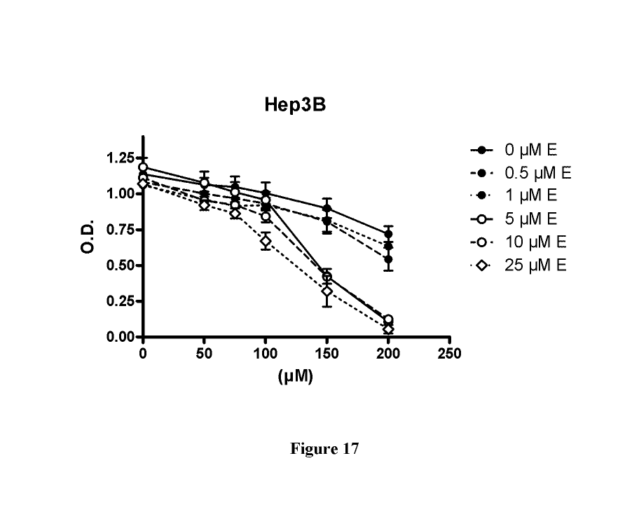

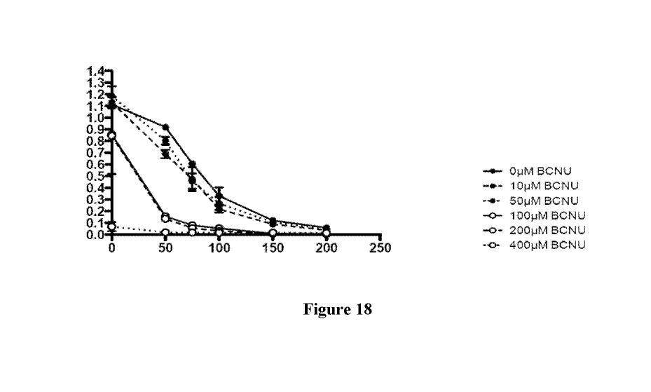

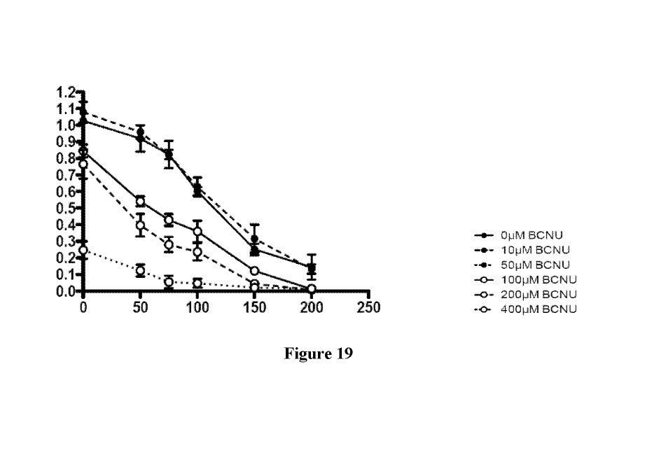

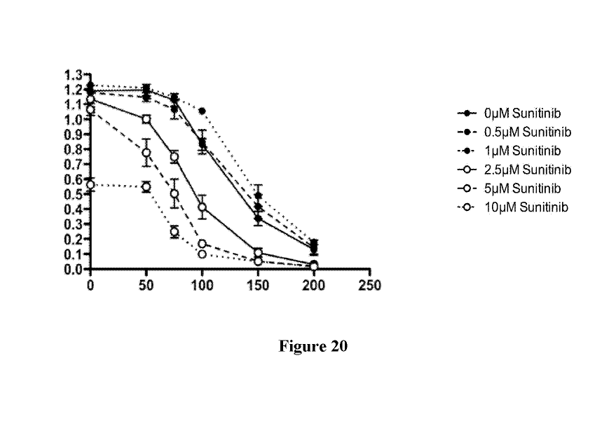

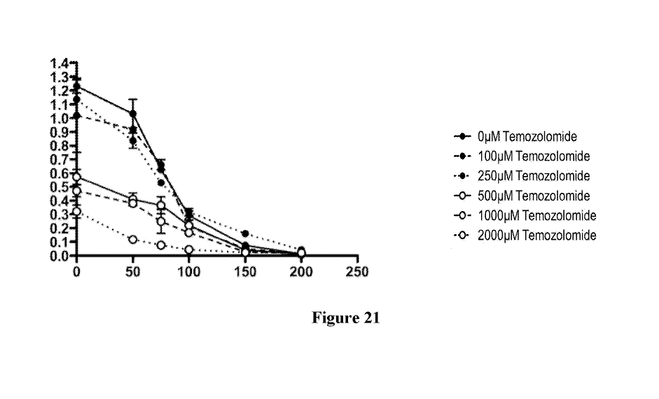

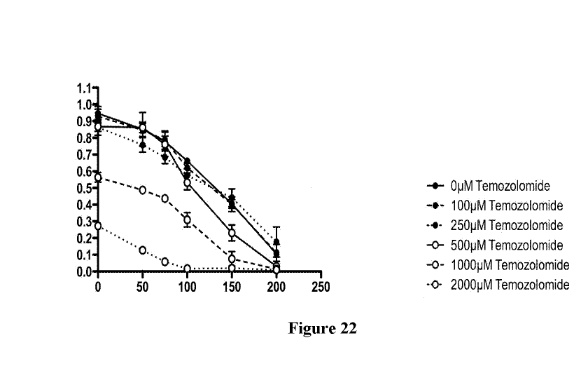

As shown in FIGS. 16-22, significant synergies were exhibited by the combination of sodium trans-[tetrachlorobis(1H-indazole)ruthenate(III)] and anti-cancer drugs including erlotinib (FIGS. 16 and 17), BCNU (FIGS. 18 and 19), sunitinib (FIG. 20), and temozolomide (FIGS. 21 and 22) in the cell lines tested.

### EXAMPLE 3

Sorafenib was purchased from LC Laboratories (Woburn, USA). All other substances were purchased from Sigma-Aldrich (St. Louis, USA).

Cell Culture: The hepatocellular carcinoma cell line Hep3B was purchased from American Type Culture Collection, Manassas, Va. Cells were grown in RMPI 1640 supplemented with 10% fetal bovine serum. The colon carcinoma cell line HCT116 and respective subline with deleted p53 genes were grown in McCoy's culture medium supplemented with 10% FCS. See Bunz et al, Cancer Res., 62:1129-1133 (2002). Lung cancer cell line A549 was grown in RPMI 1640 medium with 10% FCS, and the hepatocellular carcinoma cell line HepG2 was cultured in the Minimal Essential Medium supplemented with non-essential aminoacids, pyruvate, and 10% FCS. Lung carcinoma cell line VL-8 established in the Institute of Cancer Research, Vienna was grown in RPMI 1640 medium supplemented with 10% FCS. See Berger et al., Int. J. Cancer, 73:84-93 (1997). The mesothelioma cell model P31 and its respective cisplatin-resistant subline P31/cis was grown in Eagle's minimal essential medium with 10% FCS. See Janson et al., Cell Physiol. Biochem., 22:45-56 (2008). Cultures were regularly checked for Mycoplasma contamination.

Cytotoxicity Assays: Cells were plated (2×10₃ cells in 100 μl/well) in 96-well plates and allowed to recover for 24 hours. Drugs were added in another 100 μl growth medium and cells exposed for 72 hours. The proportion of viable cells was determined by MTT assay following the manufacturer's recommendations (EZ4U, Biomedica, Vienna, Austria).

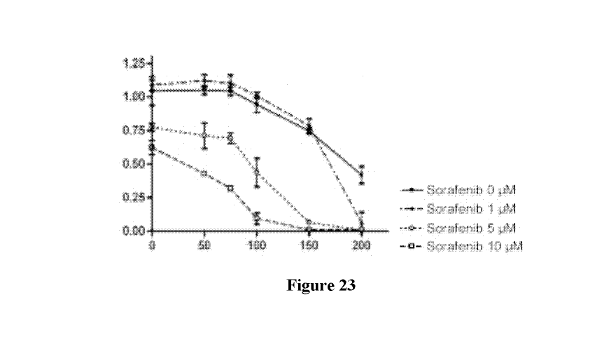

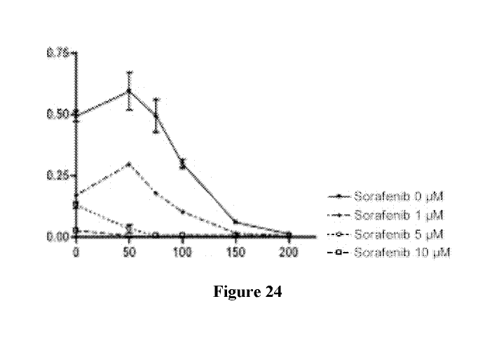

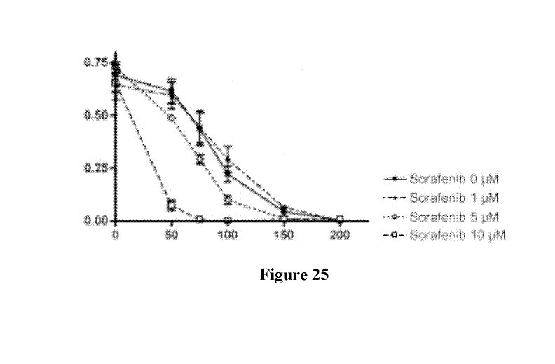

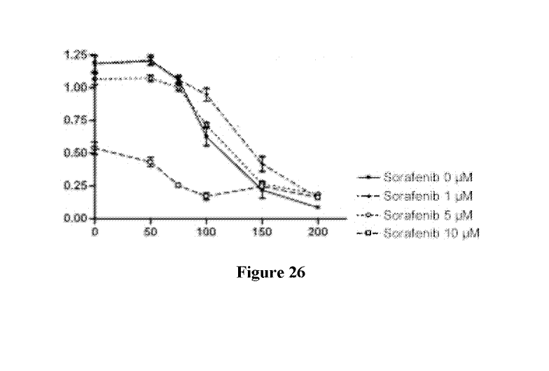

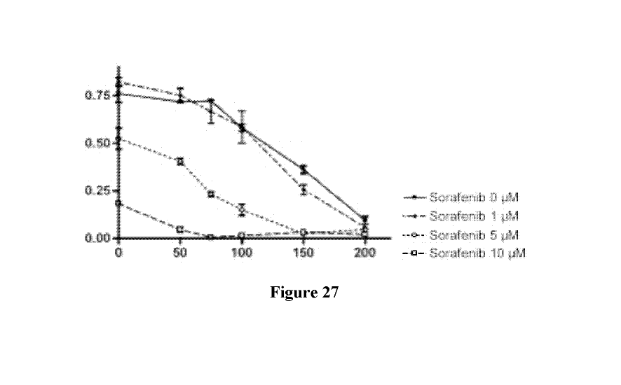

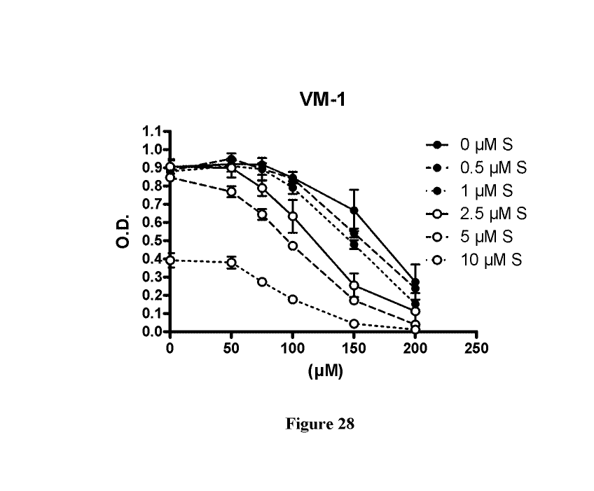

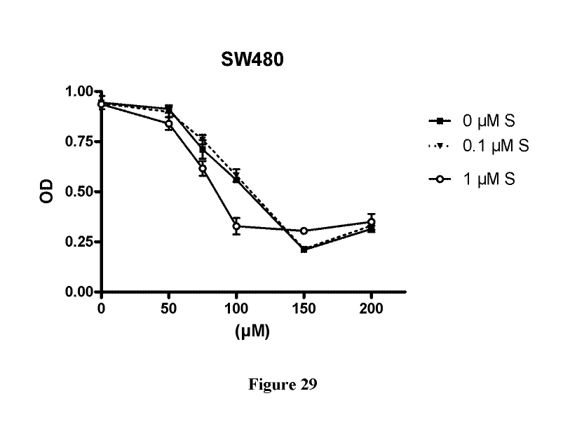

As shown in FIGS. 23-29, the combination of sorafenib and sodium trans-[tetrachlorobis(1H-indazole)ruthenate(III)] gives rise to significant synergies in a variety of cell lines including hepatocellular carcinoma cell line Hep3B, hepatocellular carcinoma cell line HepG2, lung carcinoma cell line VL-8, lung carcinoma cell line A549, mesothelioma cell line P31, colon cancer cell line SW480, and melanoma cell line VM-1.

Xenograft Assay: CB17 severe combined immunodeficient (SCID) female mice were used for all in vivo studies. The mice received food and water ad libitum. For tumor application, logarithmically growing Hep3B cells in cell culture were collected by trypsinization and washed once in serum-free culture medium. The cells were then pelleted and resuspended in culture medium to a final cell count of 2×10⁷/ml. 50 μl of the cell suspension was injected s.c. in the right flank of each mouse. Treatment started when all animals in the study had established tumors of a size of about 3×3 mm.

Sodium trans-[tetrachlorobis(1H-indazole)ruthenate(III)] was administered i.v. at a final concentration of 30 mg/kg body weight once a week for 2 weeks (day 1 and day 8). Sorafenib (LC Laboratories, Woburn, Mass., USA) was dissolved in DMSO (50 mg/ml), further diluted in Cremophor EL/95% ethanol (50:50; Sigma), which was followed by a 1:4 dilution in water. 100 μl sorafenib was administered p.o. once daily at 5 consecutive days for two weeks at a dose of 25 mg/kg body weight (days 1-5 and days 8-12).

Tumor size was calculated using the equation (l×w²)/2, where l and w refer to the larger and smaller dimensions, respectively, of the tumor. 4 mice were used in each group for each data point.

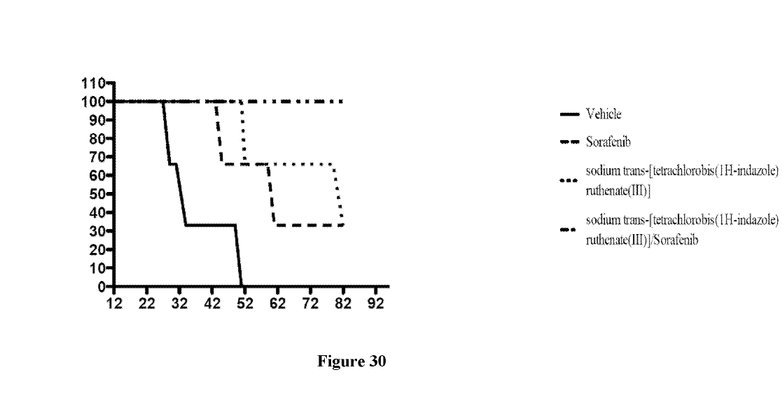

Sodium trans-[tetrachlorobis(1H-indazole)ruthenate(III)] treatment as a single agent led to a 2.4-fold increase in life span (mean survival 80 days vs. 33 days in control) and thus was superior to sorafenib monotherapy, which induced a 1.9-fold survival increase (60 days). Combination of sodium trans-[tetrachlorobis(1H-indazole)ruthenate(III)] with sorafenib increased the mean survival by 3.9-fold to 96 days. See FIG. 30.

### EXAMPLE 4

The purpose of this experiment was to evaluate the efficacy of sodium trans-[tetrachlorobis(1H-indazole)ruthenate(III)], administered intravenously (IV) as a single agent and in combination with cisplatin against early stage N87 human gastric carcinoma xenografts in female nude mice.

Female athymic mice (Hsd: Athymic Nude-Foxn1nu) were obtained from Harlan. They were 8 weeks old on Day 1 of the experiment. The mice were fed irradiated Rodent Diet 5053 (LabDiet™) and water ad libitum, and grown and experimented on in a clean and controlled environment. Test mice were implanted subcutaneously on Day 0 with 30 to 60 mg tumor fragments. All mice were observed for clinical signs at least once daily. Mice with tumors in excess of 1 g or with ulcerated tumors were euthanized. All procedures carried out in this experiment were conducted in compliance with all the laws, regulations and guidelines of the National Institutes of Health (NIH) and with the necessary approvals.

Treatments began on Day 3. All mice weighed ≧18.2 g at the initiation of therapy. Mean group body weights at first treatment were well-matched (range of group means, 22.3-22.8 g). All mice were dosed according to individual body weight on the day of treatment (0.2 ml/20 g). Sixteen days after the initial course of treatment was completed, a second course of treatment was begun for the combination groups only (groups in which cisplatin was dosed at 7.5 mg/kg). A complete second course of sodium trans-[tetrachlorobis(1H-indazole)ruthenate(III)] was given, but only two of the three planned doses of cisplatin were completed due to extensive weight loss.

Body weights and tumor measurements were recorded twice weekly. Tumor burden (mg) was estimated from caliper measurements by the formula for the volume of a prolate ellipsoid assuming unit density as: Tumor burden (mg)=(L×W2)/2, where L and W are the respective orthogonal tumor length and width measurements (mm). The primary endpoints used to evaluate efficacy were: % T/C, tumor growth delay, and the number of tumor-free survivors (TFS) at the end of the study. % T/C is defined as the median tumor mass of the Treated Group divided by the median tumor mass of the Control Group×100. In this experiment, % T/C was evaluated when the median Control reached 1 g. Tumor Growth Delay (T−C) was also used to quantify efficacy. Tumor growth delay for this experiment was expressed as a T−C value, where T and C are the median times in days required for the treatment and control group tumors, respectively, to grow to a selected evaluation size, 750 mg.

RESULTS: In this experiment, tumor growth delay and Day 28% T/C values (when the medium tumor mass in the Vehicle control Group surpassed 1 g) were used to evaluate the anti-cancer activity of the tested compounds. Treatment with sodium trans-[tetrachlorobis(1H-indazole)ruthenate(III)] (“test drug”) at 30 mg/kg (on days 3, 5, 7, and 27, 29, 31) plus cisplatin at 7.5 mg/kg (on days 3, 7, 11, and 27, 31) produced a significant (P<0.05) tumor growth delay of 16.2 days and a Day 28 T/C value of 16%. The difference in tumor growth delays between the combination regimen and the single agent regimens was significant.

### EXAMPLE 5

The purpose of this experiment was to evaluate the efficacy of sodium trans-[tetrachlorobis(1H-indazole)ruthenate(III)] as a single agent and in combination with paclitaxel against early stage A549 human lung carcinoma xenografts in female nude mice. Sodium trans-[tetrachlorobis(1H-indazole)ruthenate(III)] was administered intravenously every two days for three treatments and paclitaxel was administered intravenously for five consecutive days, both beginning on Day 3 post implant. The animals were grown, implanted with tumors and experimented on in the same manner as in Example 5 above, unless otherwise clarified below.

Cremophor EL® was used in the context of paclitaxel administration. Specifically, on each day of treatment, the paclitaxel was dissolved in absolute ethanol (10% of the final volume), followed by sequential addition of Cremophor EL® (10% of the final volume) and saline (80% of the final volume) with thorough mixing after each addition.

Treatments began on Day 3. All mice weighed ≧17.3 g at the initiation of therapy. Mean group body weights at first treatment were well-matched (range of group means, 20.6-23.5 g). All mice were dosed according to individual body weight on the day of treatment (0.2 ml/20 g).

RESULTS: In this experiment, tumor growth delay and Day 38 (the day that the mean tumor burden of the Control group surpassed the evaluation size of 1 g) % T/C values were used to evaluate anti-cancer activity. Treatment with sodium trans-[tetrachlorobis(1H-indazole)ruthenate(III)] at 30 mg/kg (on days 3, 5, and 7) plus paclitaxel at 20 mg/kg (on days 3, 4, 5, 6, and 7) produced a significant (P<0.05) tumor growth delay of 16.1 days and a Day 38 T/C value of 37% that was also significant.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method of treating cancer, comprising:
administering to a patient in need of treatment, simultaneously or sequentially (1) a pharmaceutically acceptable salt of trans-[tetrachlorobis(1H-indazole)ruthenate(III)], and (2) one or more anti-cancer agents chosen from the group consisting of platinum agents, taxane, anthracyclines, 5-FU and prodrugs thereof, nitrosourea compounds, gemcitabine, temozolomide, EGFR inhibitors, mTOR inhibitors, sorafenib, regorafenib, and sunitinib.

2. The method of claim 1, wherein said pharmaceutically acceptable salt of trans-[tetrachlorobis(1H-indazole)ruthenate(III)] is sodium trans-[tetrachlorobis(1H-indazole)ruthenate(III)].

3. The method of claim 2, wherein said one or more anti-cancer agents includes a platinum agent.

4. The method of claim 3, wherein said platinum agent is cisplatin, carboplatin, or oxaliplatin.

5. The method of claim 2, wherein said one or more anti-cancer agents includes an anthracycline.

6. The method of claim 5, wherein said anthracycline is doxorubicin.

7. The method of claim 2, wherein said one or more anti-cancer agents includes 5-FU or a prodrug thereof.

8. The method of claim 2, wherein said one or more anti-cancer agents includes a nitrosourea compound.

9. The method of claim 8, wherein said nitrosourea compound is BCNU.

10. The method of claim 2, wherein said one or more anti-cancer agents includes gemcitabine.

11. The method of claim 2, wherein said one or more anti-cancer agents includes temozolomide.

12. The method of claim 2, wherein said one or more anti-cancer agents includes an EGFR inhibitor.

13. The method of claim 12, wherein said EGFR inhibitor is erlotinib.

14. The method of claim 2, wherein said one or more anti-cancer agents includes an mTOR inhibitor.

15. The method of claim 14, wherein said mTOR inhibitor is everolimus.

16. The method of claim 2, wherein said one or more anti-cancer agents includes sorafenib or regorafenib.

17. The method of claim 2, wherein said one or more anti-cancer agents includes sunitinib.

18. The method of claim 2, wherein said one or more anti-cancer agents includes a taxane.

19. The method of claim 18, wherein said taxane is docetaxel or paclitaxel.

20. A kit, comprising in a compartmentalized container:
a first unit dosage form of a pharmaceutically acceptable salt of trans-[tetrachlorobis(1H-indazole)ruthenate(III)]; and
a second unit dosage form of one anti-cancer agent chosen from the group consisting of platinum agents, taxane,anthracyclines, 5-FU and prodrugs thereof, nitrosourea compounds, gemcitabine, temozolomide, EGFR inhibitors, mTOR inhibitors, sorafenib, regorafenib, and sunitinib.
